(19) United States
(12) Patent Application Publication
(10) Pub. No.: US 2010/0029000 A1
(43) Pub. Date: Feb. 4, 2010

(54) Assembly and methods for cell or tissue culture and treatment

(76) Inventors: Shilong Zhong, Guangzhou (CN); Hong Wu, Guangzhou (CN)

Correspondence Address: SCI HEAD PTO;Hui-Hua Commercial trading building, Room 1508, No. 80, Shen-Lei Chung Road, Guangzhou City, 510070, CN

(21) Appl. No.: 12/453,095

(22) Filed: Apr. 29, 2009

(30) Foreign Application Priority Data
Apr. 30, 2008 (CN) 200810027835.0

(51) Int. Cl. C12N 5/02; C12M 1/00

(52) U.S. Cl. 435/383; 435/303.1

(57) ABSTRACT

The invention provides an assembly designed under the multiwell plate make and methods of using the same for dynamic-based cell culture and pharmacokinetic-based cell treatment. The assembly comprises a multiwell plate and a lid which permits continuously adding and removing culture medium for culturing cells or tissues in vitro. The dynamic medium makes it possible to culture cells or tissues exposing to changing concentration of a drug candidate over time.

### FIELD OF THE INVENTION

This invention relates to a culture vessel assembly comprising a multiwell plate and a lid which permits continuously adding and removing culture medium for growing and treating cells or tissues in vitro.

### BACKGROUND OF THE INVENTION

Cells are cultured in vitro for a variety of purposes, including cell engineering, basic biochemical and cell biology research to further understand natural biological processes. In the area of drug discovery and drug development, cell culture systems are used extensively for the evaluation of pharmacological and toxicological effects of drug candidates.

Traditional method for cell culturing is to place living cells in the culture wells of plate with a fixed medium for a fixed period. The usual method for determining the effects of compounds on cells or tissues is generally to expose a cultured cell or tissue to a constant or static concentration of one or more drugs for a fixed period.

However, such an in vitro system may not accurately predict in vivo effects, because, in vivo cells live in dynamic medium environment, wherein cells can get nutrients from fresh medium, fresh medium then becomes spent medium, and the waste generated by cells is then brought away through spent medium. More particularly, during the clinical treatment of diseases in patients, cells are exposed to a continuously changing concentration of drugs.

In order to accurately predict in vivo effects, in vitro culture conditions must mimic the in vivo conditions to a large extent. Dynamic culturing by continuously adding fresh medium to cell culture well and continuously removing spent medium from culture well is a good method for this purpose. It is because dynamic culturing can supply cell with optimum nutrient, appropriate oxygen, and remove waste such as metabolic products and cell debris. Further more, dynamic culture can continuously dilute drug concentration and therefore are able to duplicate the pharmacokinetics of dose regimens in living being where concentrations are greatest immediately following exposure and decline with time.

There are a number of culture devices related to dynamic culturing commercially available and described in patent publications. For example, U.S. Patent 20070037273 to Shuler, et al, published on Feb. 15, 2007 describes devices permitting cells to be maintained in vitro, under conditions with pharmacokinetic parameter values similar to those found in vivo. The apparatus described by Diresta et al, U.S. Patent 20030211600, published on Nov. 11, 2003, may also be used with fluid replenishment for growing cells. U.S. Pat. No. 6,576,458 to Sarem, et al, published on Jun. 10, 2003, describes a cell and tissue culture device in which the culture fluid can be set into motion and achieve a dynamic culture.

However, these devices are developed not based on the standard multiwell plate which is most commonly used for cell culture and ideal device for high throughput screening. They are therefore not suitable for high throughput screening for evaluating cell function or drug effect. Furthermore, they are often complex and cumbersome which make them very costly.

In order to evaluate cell function or drug effect in a cost effective and high throughput, it is ideal to use an in vitro device designed under the multiwell plate make for dynamic culturing and treatment.

### SUMMARY OF THE INVENTION

The object of the invention is therefore to provide an assembly and methods for growing and/or treating cells in which the culture fluid is set into motion so as to achieve a dynamic culture. Such an assembly can be modeled under the multiwell plate make, which make it possible to conduct in a high throughput manner. It is therefore better suited to the large research scale and the industrial scale.

According to the invention, this object is achieved with the features of claim 1 for an assembly, and with the features of claims 11, 12, 13 and 12 for the methods. Advantageous embodiments of the invention result from the features mentioned in the subordinate claims.

The plate and related removable lid may be formed in different sizes and geometric configurations so as to be used with different size and geometric configured cell culture inserts. The removable lid may be formed to be positioned over the upper surface of the plate in one orientation so as to reduce cross contamination between the wells in the event the lid is repositioned over the upper surface of the plate. The plate and the removable lid are preferably made of an optically clear plastic to facilitate viewing of the wells and cell culture inserts.

The slots or holes in the wall of the well of the plate, the channels of the plate, and the inlets and the outlets of the lid may be formed in different sized and geometric configurations to achieve the optimization of the flow pattern.

Preferably, the lid comprises inlets for allowing medium into the compartment of lid and outlets for distributing the medium in the compartment of lid into the wells of the plate. Most desirably, each of said outlets for distributing medium into the wells of the plate has at least one side open such that reduces impact of flow on cells. Most preferably, the lid comprises the same number of outlets as the number of wells in the companion plate. In addition, most preferably, the outlets are positioned or aligned with the wells. Therefore, when the lid is placed over the plate, each well is associated with an outlet on the lid.

Preferably, the wall of the wells of the plate comprises at least one longitudinal slot or hole for medium overflow. In another embodiment, spent medium in the wells can overflow through the top edge of the wall of the wells of the plate to the upper inner surface of the body of the plate.

The upper inner surface of the body of the plate may be formed in different geometric configurations for effectively draining medium. The inclined upper inner surface is one of embodiments. Preferably, the channels, holes, or orifices in the plate locates at the lowest position of the inclined inner surface so as to effectively drain medium overflowed from the longitudinal slots, or holes in or the top edge of the well.

In a preferred embodiment, medium flow is driven by an external pump which can control flow rate. In another embodiment, medium flow is driven by the gravity of medium as the source medium is placed higher than the position of lid.

In an advantageous embodiment of the invention, it is possible to continuously introduce fresh culture medium into the cell culture wells from the compartment of the lid through outlets of the lid and remove spent medium from culture well through outlet in the wall of well.

In another advantageous embodiment of the invention, it is possible to grow culture exposing to changing concentration of a drug(s) which simulates. in vivo pharmacokinetic process.

### DESCRIPTION OF THE DRAWINGS

FIG. 1 is a top exploded view of the assembly of the present invention.

FIG. 2 is a bottom view of the lid of this invention.

FIG. 3 is a partial sectional view of the outlet of the lid.

FIG. 4 is a top view of a multiwell plate of this invention.

FIG. 5 is a partial sectional view of the well of the plate.

FIG. 6 is a partial sectional view of the outlet of the plate.

FIG. 7 is a cross-sectional view of the plate and the lid.

FIG. 8 is a partial cross-sectional view of the wells of the plate and the outlets of the lid.

### DETAILED DESCRIPTION OF SPECIFIC EMBODIMENTS

While this invention is satisfied by embodiments in many different forms, there is shown in the drawings and will herein be described in detail, the preferred embodiments of the invention, with the understanding that the present disclosure is to be considered as exemplary of the principles of the invention and is not intended to limit the invention to the embodiments illustrated. Various other modifications will be apparent to and readily made by those skilled in the art without departing from the scope and spirit of the invention. The scope of the invention will be measured by the appended claims and their equivalents.

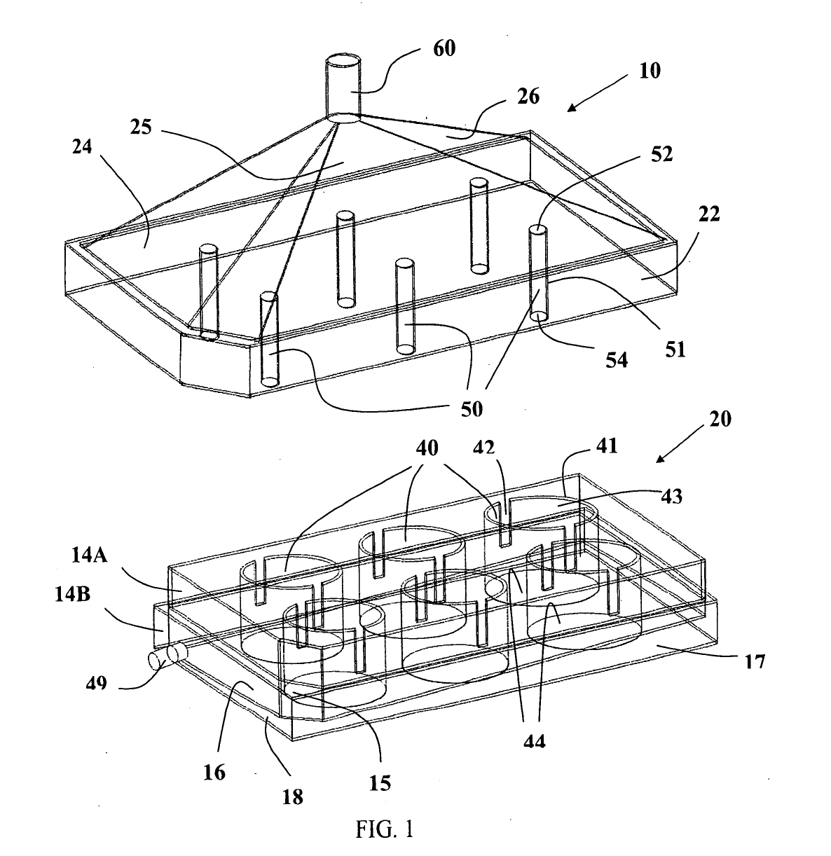

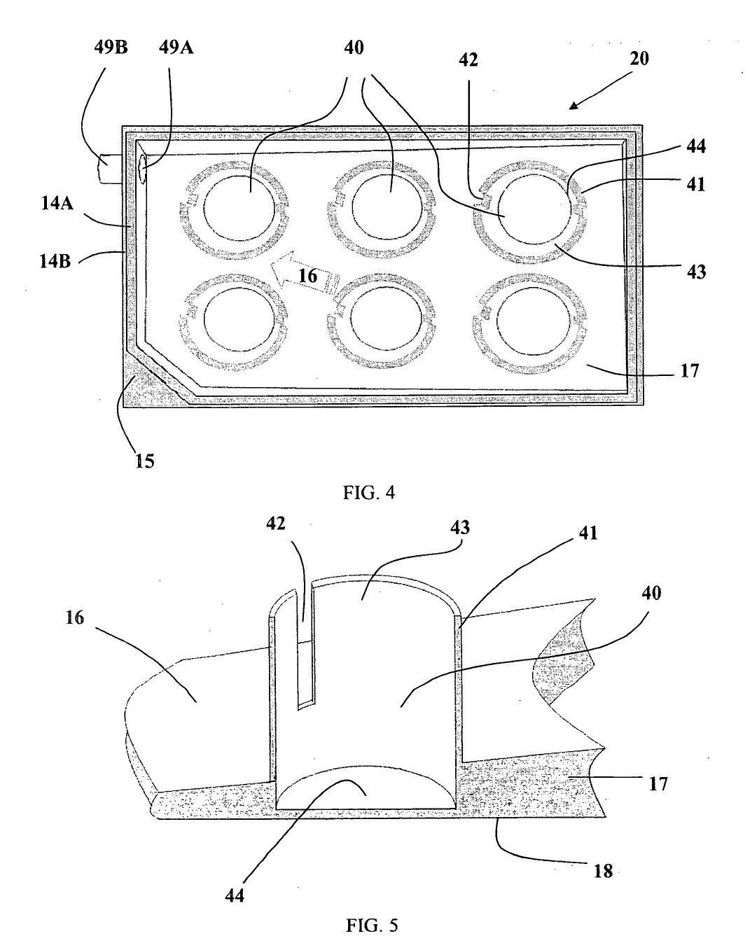

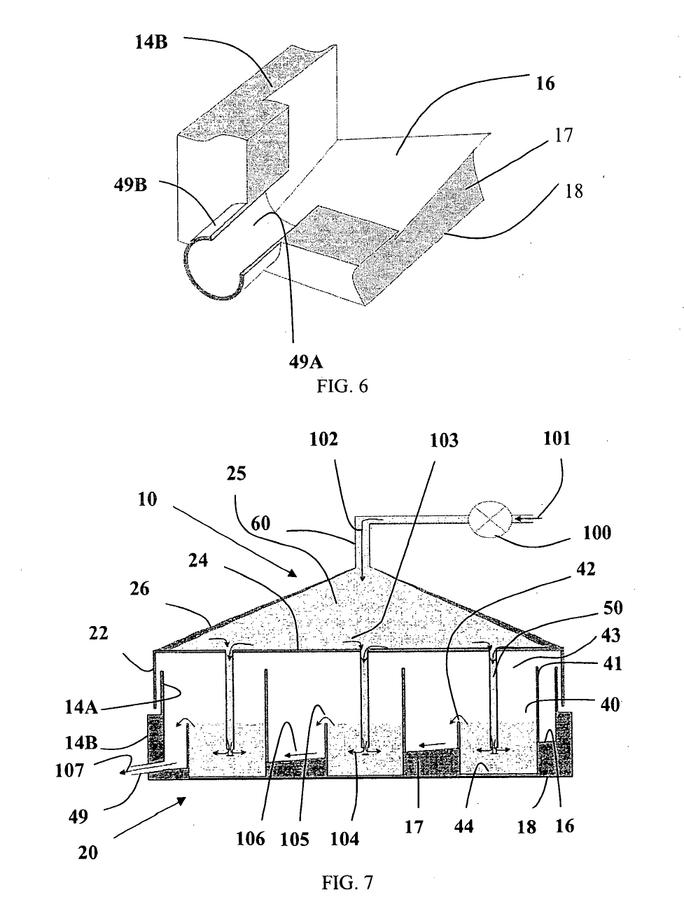

Referring to FIG. 1, the assembly of this invention comprises a plate 20 and a lid 10. Also as shown in FIG. 4, the plate 20 includes a body 17, a plurality of wells 40, upstanding sidewalls 14 and a channel 49. The upstanding sidewall 14 comprises a two-step section comprising an upper section 14A and a lower section 14B. The lower section 14B is stepped from the upper section 14A by an annular shoulder 15 between the upper section and lower sections. Lower section 14B comprises four sides. Upper section 14A comprises five sides. The channel 49 comprises a hole 49A and a wall 49B.

Referring to FIGS. 1, 4, 5, 7 and 8, Well 40 comprises a fluid impervious wall 41, an open top 43 and a bottom 44. The wells can be in the same height from open top 43 to bottom 44 and the bottoms of wells is parallel to outer surface of said plate. In order to drain medium efficiently, the distance of the bottom 44 of well 40 from lower outer surface 18 of the body 17 of the plate 20 can be different from each other. In the present embodiment, the distance of the bottom 44 of well 40 from lower outer surface 18 of the body 17 of the plate 20 is the same to each other, as shown in FIG. 7, The well 40 may be formed in a variety of shapes, but tubular designs are preferred for such wells. As best seen in FIG. 5, in one embodiment, Wall 41 further comprises at least one slot 42 open to the top of the wall 41. Such slots facilitate spent medium overflowing from well. The size, location and number of slot 42 may be varied along the wall 41 to optimize the flow pattern.

The body 17 of the plate 20 further comprises an inclined upper inner surface 16 and a horizontal orientation lower outer surface 18. The upper inner surface 16 is formed inclined so as to drain effectively spent medium overflowed from slot 42 to the hole 49. The inner surface of the plate can be molded to be inclined or depress to form a drain area. The present embodiment of the inner surface 16 is inclined. The lowest part of the inner surface connects the outlet 49.

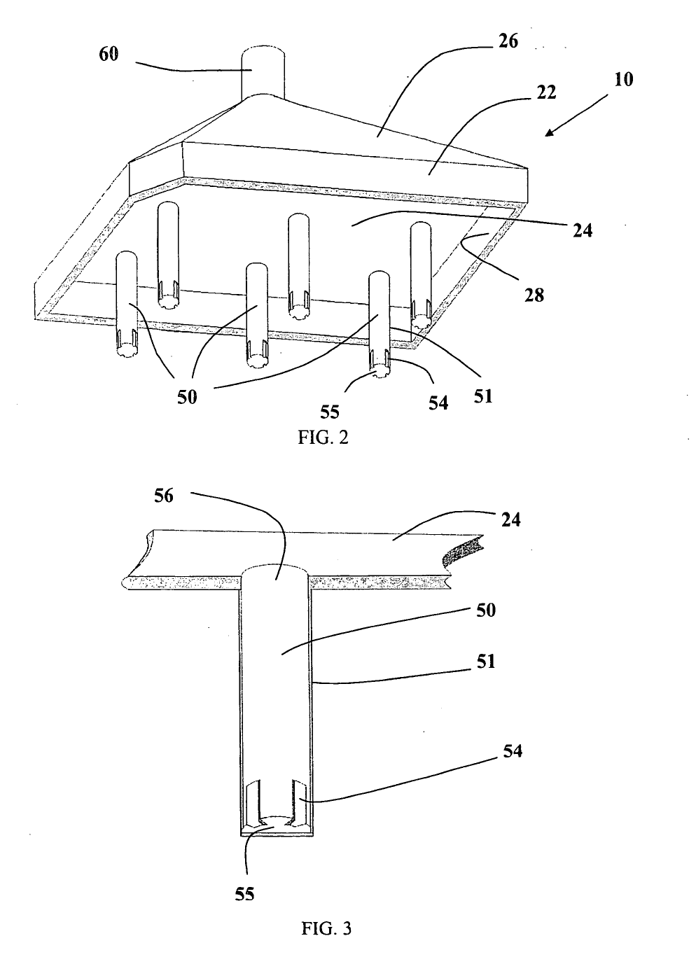

The lid serves to receive medium through inlets and distribute medium through outlets to the wells of the plate. The inlets are provided with a connection with external pump for supplying the cell culture well of the plate with culture medium or drug solution. Although external pump is preferable for controlling medium flow, the self's weight of medium can also be used to drive medium flow when the position of medium source is located higher than that of lid. FIGS. 1 and 2 illustrate one embodiment of the lid of the assembly according to the invention. The lid 10 comprises an upper section 26, a lower section 24, a fluid reservoir compartment 25 between the upper section 26 and the lower section 24, an inlet 60 extending from the upper section 26 and serving as a medium entry for the compartment 25, outlets 50 extending from the lower section 24 for distributing medium to the wells 40 of plate 20, and a skirt 22 surrounding the lower section defining a plurality of corners on the lid 10 and extending downwardly. Under the spirit of current invention, practitioners-in-the-art will understand the shape of the upper section 26 may be concave surface, planar surface, or other configuration. The position of inlet may be located at any part of lid. The number of inlet may be more than one. Take the lid with two inlets for an example, one inlet may serve to receive medium, the other one may serve to receive drug solution. The size of the compartment between the upper section 26 and the lower section 24 can be varied.

In one embodiment, the lid may be molded the upper section and the lower section together, with a compartment between the upper section and the lower section. In another embodiment, the lid may be assembled from an upper section and a lower section. A fluid reservoir compartment is formed when the upper section and the lower section are assembled. At least one inlet extends from the upper section or the lower section and serves as a medium entry for the compartment. At least one outlet extends from the lower section of the lid for distributing medium. A skirt surrounds the lower section defining a plurality of comers on the lid and extending downwardly.

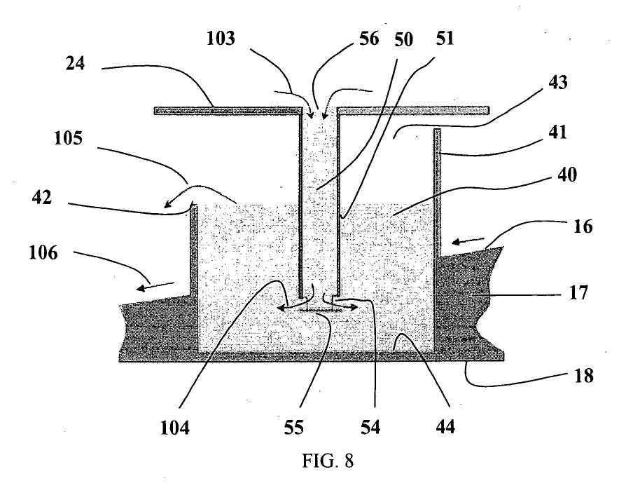

FIGS. 1, 2, 3, 7, and 8 illustrate one embodiment of the outlets of the lid according to the invention. The outlet 50 includes a fluid impervious wall 51, an open top 56 and at least one hole, slot or orifice 54. As best seen in FIGS. 3 and 8, most preferably, the outlet further includes a bottom 55, which can reduce the impact of medium flow on cultured cells. The shape. of the outlet 50 may be circular, rectangular, or other configurations.

As shown in FIG. 1 and FIG. 7, the configuration of lid 10 is compatible with that of plate 20. Both the plate 20 and the skirt 22 of the lid 10 comprise five sidewalls, which are made to align the lid and the plate of the assembly such that the outlets 50 of lid 10 can insert into the matched wells 40 of plate 20 precisely and consistently. Although the position of the hole 54 of the outlets 50 can be higher than the position of the slot 42 of the wells 40, in order to achieve constant medium flow and reduce the impact of flow on cultured cell, the position of the hole 54 of the outlets 50 can be below the position of the slot 42 of the wells 40 and the difference between the bottom 55 of the outlets 50 and the bottom 44 of the wells 40 can be more than 1 micrometer when the lid 10 is resting on the plate 20 and the outlets 50 of lid 10 insert into the wells 40 the plate 20.

In FIG. 1, a six-well plate 20 and matched six-outlet lid 10 are illustrated, merely for exemplary purposes. The number of the wells and outlets can be six, twelve, twenty-four, forty-eight, ninety-six, or any number. The wells of plate and matched outlet of lid may be arranged in orthogonal rows and columns or other arrangements. The plate and the lid can be circular, rectangular, or other practical configurations.

In a preferred embodiment, the culture vessel assembly of the present invention is transparent and may be molded from a variety of materials, including, for example, polyethylene, polystyrene, polyethylene terephthalate and polypropylene.

Referring to FIGS. 7, there is illustrated a cross-sectional view of the assembly of the present invention. As embodied in FIG. 7, fresh medium is driven by an external pump 100 into the compartment 25 of lid 10 through the inlet 60 of the upper section 26 of the lid 10 and then is distributed into wells 40 of the plate 20 through the outlets 50 of the low section 24 of the lid 10. The spent medium in well 40 overflows through slot 42 in the wall 41 of the well 40 to the declined upper inner surface 16 of plate 20. Spent medium on the declined upper inner surface 16 is drained away through the channel 49. The medium overflows from the wells 40 at the same rate as the medium is added. In one embodiment, the medium flow pathway is from 101, to 102, to 103, to 104, to 105, to 106, and to 107.

The cell culture assembly of the present invention allows a researcher to culture cells on the bottom of well 40. Fresh medium for cells growing are then supplied continuously from the lid and spent medium in the wells is removed through the plate drain system. The plate drain system includes the slot 42 in the wall 41 of well 40, the declined inner surface 16 of the body 17, the channel 49 in the plate 20.

One of the main advantages of the present invention lies in the dynamic system with continuously adding fresh medium to cell culture wells from the compartment of the lid through the outlets of the lid and continuously removing spent medium from culture wells through the plate drain system. The flow rate can be controlled by external pump according to experiment design.

Another of the main advantages of the present invention lies in the basis of multiwell plate make. Every well mimics an in vivo dynamic system. Therefore, the invention can be used as a high-throughput dynamic system for the study in the physiologic or pathologic conditions.

One of the main areas of use of the method according to the invention is therefore the investigation of the action of drug candidates on cells or tissues. In operation, cells or tissues are cultured in the wells with the dynamic medium controlled by an external pump. Drug candidates are then added into the wells and removed over time by controlled adding drug free fresh medium and removing spent medium. The medium flow rate can be calculated based on pharmacokinetics of one compartment model and controlled by a pump system to mimic the drug elimination half-life observed in vivo. After treatment, different parameters about treated cells or tissue are measured to determine the drugs effectiveness.

The assembly of the present invention is also useful in the area of cell or tissue engineering.

What is claimed is:

1. An assembly comprising:
a plate comprising, a body comprising an upper inner surface and a lower outer surface, at least one well extending from said body, upstanding sidewalls forming an outside border of the plate, at least one channel extending from said upper inner surface of said body to the outside of the plate for removing medium from said upper inner surface; and
a removable lid positioned on said plate over said wells comprising, an upper section, a lower section, a compartment between said upper section and said lower section, at least one inlet which serves as a fluid entry for said compartment, at least one outlet extending from said lower section for distributing fluid to the well of said plate, and a skirt surrounding said compartment defining a plurality of comers on said lid and extending downwardly having an inner and outer surface; and
means for continuously adding culture medium to the wells of the plate and continuously removing culture medium from said wells of said plate.

2. The plate of claim 1, wherein said upper inner surface of said body is inclined so as to effectively remove medium overflowed from said well to said outlet in said plate.

3. The plate of claim 1, wherein said channels extend from said upper inner surface of said body to the outside of the plate through said body of said plate for removing medium from said upper inner surface.

4. The plate of claim 1, wherein said channels extend from said upper inner surface of said body to the outside of the plate through upstanding sidewalls of said plate for removing medium from said upper inner surface.

5. The plate of claim 1, wherein each of said wells further comprise,
a bottom; and
an open top; and
a fluid impervious wall comprising at least one outlet through which culture medium can overflow to the inner upper surface of said body.

6. The plate of claim 5, wherein said outlets in said wall are slots.

7. The plate of claim 5, wherein said outlets in said wall are holes.

8. The lid of claim 1, wherein said inlets are provided with a connection with external pump for supplying said compartment with culture medium.

9. The assembly of claim 1, wherein said outlets in said lid are precisely aligned to said well of said plate of claim 1 in order to distribute fluid to each of said well from said compartment.

10. The assembly of claim 1, wherein the position of the tips of said outlets extending from said lower section of said lid is below the position of top edge of said wall of said wells and the difference between the tip of said outlets extending from said lower section of said lid and said bottom of said wells is more than 1 millimeter when said lid is resting on said plate and said outlets of said lid insert into said wells of said plate.

11. A dynamic-based method for cell or tissue culture comprising:
(a) providing an assembly comprising a plate comprising a body comprising an declined upper inner surface and an lower outer surface, at least one well whose wall has at least one hole or slot, upstanding sidewalls forming an outside border of the plate, at least one channel extending from said upper inner surface of said body to the outside of the plate for removing medium from said upper inner surface; a removable lid positioned on said plate over said wells comprising, an upper section, a lower section, a compartment between the upper section and the lower section, at least one inlet which serves as a fluid entry for said compartment, at least one outlet extending from said lower section for distributing fluid to the well of said plate, and a skirt surrounding said compartment defining a plurality of comers on said lid and extending downwardly having an inner and outer surface;
(b) providing a method, comprising, depositing a layer of cells or tissues on the bottom of said well of said plate, positing the lid over the plate and precisely aligning said outlets extending from said lower section to said well of said plate, fresh culture medium is continuously driven into the said compartment of said lid by external pump or the self's weight of medium, fresh culture medium is continuously distributed into said wells in said plate through said outlets of said lid, fresh culture medium is spent by cells or tissues in said well and changes to spent medium, spent medium in said wells of said plate overflows through said holes or slots in said wall of said well to said upper inner surface of said plate, spent medium on said declined upper inner surface of said plate is drained to said outlets of said plate.

12. A dynamic-based method for cell or tissue culture comprising:
(a) providing an assembly comprising a plate comprising a body comprising an declined upper inner surface and an lower outer surface, at least one well, upstanding sidewalls forming an outside border of the plate, at least one channel extending from said upper inner surface of said body to the outside of the plate for removing medium from said upper inner surface, a removable lid positioned on said plate over said wells comprising, an upper section, a lower section, a compartment between the upper section and the lower section, at least one inlet which serves as a fluid entry for said compartment, at least one outlet extending from said lower section for distributing fluid to the well of said plate, and a skirt surrounding said compartment defining a plurality of comers on said lid and extending downwardly having an inner and outer surface,
(b) providing a method, comprising, depositing a layer of cells or tissues on the bottom of said well of said plate, positing the lid over the plate and precisely aligning said outlets extending from said lower section to said well of said plate, fresh culture medium is continuously driven into the said compartment of said lid by external pump or the self's weight of medium, fresh culture medium is continuously distributed into said wells in said plate through said outlets of said lid, fresh culture medium is spent by cells or tissues in said wells and changes to spent medium, spent medium in said wells of said plate overflows through the top edge of said wall of said well to said upper inner surface of said plate, spent medium on said declined upper inner surface of said plate is drained to said outlets of said plate.

13. A pharmacokinetic-based method for cell or tissue treatment comprising:
(a) providing an assembly comprising a plate comprising a body comprising an upper inner surface and an lower outer surface, at least one well whose wall has at least one hole or slot, upstanding sidewalls forming an outside border of the plate, at least one channel extending from said declined upper inner surface of said body to the outside of the plate for removing medium from said upper inner surface, a removable lid positioned on said plate over said wells comprising, an upper section, a lower section, a compartment between the upper section and the lower section, at least one inlet which serves as a fluid entry for said compartment, at least one outlet extending from said lower section for distributing fluid to the well of said plate, and a skirt surrounding said compartment defining a plurality of comers on said lid and extending downwardly having an inner and outer surface,
(b) providing a method, comprising, depositing a layer of cells or tissues on the bottom of said well of said plate, positing the lid over the plate and precisely aligning said outlets extending from said lower section to said well of said plate, fresh culture medium is continuously driven into the said compartment of said lid by external pump or the self's weight of medium, fresh culture medium is continuously distributed into said wells in said plate through said outlets of said lid, fresh culture medium is spent by cells or tissues in said wells and changes to spent medium, spent medium in said wells of said plate overflows through said holes or slots in said wall of said well to said upper inner surface of said plate, drug candidate is removed over time by controlled adding fresh medium and removing spent medium, cells or tissues are cultured over a period of time with dynamic medium by continuously providing cells in culture well with fresh culture medium from the lid and continuously removing spend culture medium through the slots of the well of the plate from culture wells, cells or tissues are treated with drug candidate, drug candidate is removed over time by controlled adding fresh medium and removing spent medium, different parameters about treated cells or tissues are measured to determine the drugs effectiveness.

14. A pharmacokinetic-based method for cell or tissue treatment comprising:
(a) providing an assembly comprising a plate comprising a body comprising an upper inner surface and an lower outer surface, at least one well, upstanding sidewalls forming an outside border of the plate, at least one channel extending from said declined upper inner surface of said body to the outside of the plate for removing medium from said upper inner surface, a removable lid positioned on said plate over said wells comprising, an upper section, a lower section, a compartment between the upper section and the lower section, at least one inlet which serves as a fluid entry for said compartment, at least one outlet extending from said lower section for distributing fluid to the well of said plate, and a skirt surrounding said compartment defining a plurality of corners on said lid and extending downwardly having an inner and outer surface,
(b) providing a method, comprising, depositing a layer of cells or tissues on the bottom of said well of said plate, positing the lid over the plate and precisely aligning said outlets extending from said lower section to said well of said plate, fresh culture medium is continuously driven into the said compartment of said lid by external pump or the self's weight of medium, fresh culture medium is continuously distributed into said wells in said plate through said outlets of said lid, fresh culture medium is spent by cells or tissues in said wells and changes to spent medium, spent medium in said wells of said plate overflows through the top edge of said wall of said well to said upper inner surface of said plate, drug candidate is removed over time by controlled adding fresh medium and removing spent medium, cells or tissues are cultured over a period of time with dynamic medium by continuously providing cells in culture well with fresh culture medium from the lid and continuously removing spend culture medium through the slots of the well of the plate from culture wells, cells or tissues are treated with drug candidate, drug candidate is removed over time by controlled adding fresh medium and removing spent medium, different parameters about treated cells or tissues are measured to determine the drugs effectiveness.
